(19) United States
(12) Patent Application Publication
(10) Pub. No.: US 2022/0241366 A1
(43) Pub. Date: Aug. 4, 2022

(54) A HERBAL MEDICINE COMPOSITION FOR TREATMENT OF DENGUE AND THEIR PRODUCTION

(71) Applicants: Pharmalytics Corporation, Gen. Trias (PH); De La Salle Medical and Health Sciences Institute, Dasmarinas (PH)

(72) Inventor: Rita Grace Y. ALVERO, Dasmarinas (PH)

(73) Assignees: Pharmalytics Corporation, Gen. Trias (PH); De La Salle Medical and Health Sciences Institute - DLSMHSI, Dasmarinas (PH)

(21) Appl. No.: 17/296,878

(22) PCT Filed: Oct. 8, 2020
(86) PCT No.: PCT/PH2020/050014

§ 371 Date: May 25, 2021

(30) Foreign Application Priority Data
Oct. 8, 2019 (PH) 12019000387

(51) Int. Cl. A61K 36/9068; A61K 36/185; A61K 36/47; A61K 9/48

(52) U.S. Cl. CPC A61K 36/9068; A61K 36/185; A61K 36/47; A61K 2236/53; A61K 2236/333; A61K 2236/17; A61K 2236/15; A61K 9/4833

(57) ABSTRACT

This invention relates to herbal medicine compositions for the treatment of dengue and their production. The herbal medicine compositions consist of dried and finely powdered leaves of Lagerstroemia speciosa; dried and finely powdered aerial parts of Euphorbia hirta; dried and finely powdered rhizome of Zingiber officinale; and a fixed-dose combination of all three. In another version of the formulation, the spray-dried aqueous, ethanolic, methanolic or hydroalcoholic extract of the mentioned plants were used instead.

### TECHNICAL FIELD

The present invention relates to a herbal medicine composition for treatment of dengue and their production.

### BACKGROUND OF THE INVENTION

Dengue is the most rapidly spreading mosquito-borne viral disease in the world. In the last 50 years, incidence has increased 30-fold with increasing geographic expansion to new countries and in the present decade from urban to rural settings. Some 1.8 billion (more than 70%) of the population at risk for dengue worldwide live in member states of the WHO South-East Asia Region and Western Pacific Region which bear nearly 75% of the current global disease burden due to dengue. There is no gender predisposition and although most cases are young children there is an increasing proportion of adults.

Dengue has a wide spectrum of clinical presentations, often with unpredictable clinical evolution and outcome. While most patients recover following a self-limiting non-severe clinical course, a small proportion progress to severe disease, mostly characterized by plasma leakage with or without haemorrhage. It has been observed that dengue is more severe in secondary infections as compared to primary infections. It is presumed that patients with prior infection manifest an enhanced immune response that leads to a more severe form of the disease. This highlights the need for a definitive drug treatment as opposed to vaccination.

There are three important stages in the cascade of events in the critical stage of dengue that can be alleviated. namely: the augmentation of viral multiplication, increased vascular permeability and reduced platelets count as illustrated in FIG. 1. Currently, there are no definitive treatments for the alleviation of such pathophysiologic conditions. However, several medicinal plants have shown promising bioactivities that could be exploited for dengue treatment.

Lagerstroemia speciosa, locally known as banaba, is a semi-deciduous tree that is native to South and Southeast Asia including the Philippines. Studies have shown that compounds from L. speciosa leaves have significant antiviral properties. A study has also particularly shown that L. speciosa leaves extract has potent anti-viral activity against dengue virus as evaluated by plaque reduction neutralization test (PRNT) in Vero cell infection model. Such antiviral activity can be exploited in decreasing the multiplication of the virus in dengue patients.

Zingiber officinale, locally known as luya in the Philippines, is a widely cultivated spice for its strongly aromatic rhizomes. It has a plethora of bioactivity including anti-inflammatory and anti-viral properties. Like L. speciosa leaves, Z. officinale extract has a potent anti-viral activity against dengue virus as evaluated by PNRT in Vero cell infection model. Another bioactivity of Z. officinale that is of interest in dengue treatment is its activity against matrix metalloproteinases (MMPs). A study has shown that dengue virus-infected immature dendritic cells overproduce soluble gelatinolytic MMP-9 and MMP-2 to a lesser extent, which both promote vascular permeability. The alleviation of vascular permeability is considered as an important “battlefield” in the pathophysiology of dengue. The activity of Z. officinale against MMP-9 and MMP-2 has been widely demonstrated in various cancers. A particular study has shown MMP-9 and MMP-2 inhibition of Z. officinale extract in dengue virus-infected Vero cells by gelatin zymography. The herbal composition of Z. officinale can therefore be exploited both for decreasing viral multiplication and alleviation of vascular permeability in dengue patients.

Euphorbia hirta, locally known as tawa-tawa or gatas-gatas in the Philippines, has been traditionally used in dengue treatment for its touted ability in elevating platelet count. An increasing number of studies scientifically support such claim. In one study, the lyophilized decoction of E. hirta was able to significantly increase the platelet count in ethanol-induced thrombocytopenic rat model. In another animal model, E. hirta extract was demonstrated to increase platelet count by 80.92%. A clinical study also reveals that 70% of patients show improvement in platelet count, total leukocyte count (TLC), fever, and flu-like symptoms using E. hirta herbal water. The platelet elevating property of E. hirta can therefore be exploited to alleviate thrombocytopenia in dengue patients.

### SUMMARY OF THE INVENTION

The herbal medicine compositions are produced using dried and finely powdered leaves of Lagerstroemia speciosa; dried and finely powdered aerial parts of Euphorbia hirta; dried and finely powdered rhizome of Zingiber officinale; and a fixed-dose combination of all three. The production involves the use of air-drying and/or commercial dehydrator to reduce the moisture content of the plant samples below 10%. The dried samples were then powdered using a commercial blender or mill and the powders were then sieved. Particles that pass through No. 60 mesh sieve were then encapsulated in 500 mg dose and 400 mg dose using suitable capsule such as gelatin. In another version of the formulation, the spray-dried aqueous, ethanolic, methanol or hydroalcoholic extract (70-90% alcohol by volume) of the mentioned plants were used instead.

Various characterization and quality control tests were carried out to ensure that the product meets pharmacopeial standards. Pre-clinical tests were also carried out to evaluate the safety and efficacy of the herbal formulations. Clinical studies on the tolerability, safety and efficacy and of the formulated product are currently being conducted.

### DETAILED DESCRIPTION

Plant Collection and Processing

Leaves of L. speciosa, aerial parts of E. hirta and rhizomes of Z. officinale were collected from designated farm lands. Grading of the samples was conducted during the collection to ensure the good quality of raw materials. The samples were washed thoroughly to remove dust and other adhering particles.

The collected samples of L. speciosa and E. hirta were laid in drying bed and air-dried overnight or up to several days. The final stage of drying was carried out using a commercial dehydrator between 40-60° C. until moisture content falls below 10%. Meanwhile, the rhizomes of Z. officinale were peeled and cut into thin strips. They were dried using a commercial dehydrator between 40-60° C. until moisture content falls below 10%. The samples were then finely ground using a commercial blender or mill. The powdered plant samples were sieved using No. 60 mesh sieve. Powder that passed through the sieve were combined and placed in an air-tight vessel and stored at cool dry conditions and away from direct sunlight until encapsulation.

In another version of the formulation, the spray-dried aqueous, ethanolic, methanolic or hydroalcoholic extract (70-90% alcohol by volume) of the mentioned plants were used instead.

Sample Characterization and Quality Testing

Representative samples were analyzed for parameters that are specified in the Guidelines on the Registration of Herbal Medicine by the Department of Health of the Republic of the Philippines. The test procedures for the specified parameters were taken from standard pharmacopeial guides such as the Quality Control Methods of Medicinal Plant Materials by the World Health Organization. The results of such tests are attached as supplementary documents.

Sample Encapsulation

Once the samples passed the quality standards they were encapsulated separately in 500 mg dose and 400 mg dose using appropriate capsules. The fixed-dose combination of the three samples can be done in various ratios with a total dose of 500 mg and 400 mg. Excipients were not added to the formulations. The capsules may be packaged in blister form or in amber bottles of up to 30 capsules per bottle.

Uniformity, Dissolution and Stability Tests

Representative capsules will be tested for uniformity in dosage, dissolution and stability tests as specified in pharmacopeial guides such as the United States Pharmacopeia.

Pro-Clinical Studies

The safety of the dose formulations were evaluated by mutagenicity assays (Ames test and Macronucleus test), Acute toxicity in rats, and Subchronic toxicity tests in rats. Meanwhile, the proof of concept of anti-dengue virus inhibition was tested by Plaque Reduction Neutralization Test.

Ames assay and Micronucleus test are the standard mutagenicity assays that are required for the registration of herbal medicines in the Philippines as stipulated in DOH DAO no. 172 series of 2004 and International Council for Harmonisation of Technical Requirements for Pharmaceuticals for Human Use (ICH). The result of Ames test showed that all the plant samples are non-mutagenic in nature when tested on Salmonella typhimurium strains T98 and T100 as summarized in Tables 1 and 2. The non-mutagenic nature of the plant samples % we further proven by the Micronucleus test in mice as summarized in Table 3.

TABLE 1
Results of Ames mutagenicity test of L. speciosa, E. hirta, and z. officinale
on Salmonella typhimurium strain T98

| | | No. of Revernant | |
|---|---|---|---|
| Sample | Concentration | Colonies per plate | Mutegenicity |
| Positive control | 6 μg/plate | 69.2 ± 10.62 | Mutagenic |
| (Daunomycin) | | | |
| Spontaneous control | — | 41.8 ± 6.98 | Non-mutagenic |
| Negative control | As is | 40.6 ± 6.58 | Non-mutagenic |
| (purified distilled | | | |
| water) | | | |
| E. hirta | 250 mg/ml in | 43.6 ± 5.37 | Non-mutagenic |
| | distilled water | | |
| L. speciosa | 250 mg/ml in | 42.6 ± 10.81 | Non-mutagenic |
| | distilled water | | |
| Z. officinale | 250 mg/ml in | 43.0 ± 9.77 | Non-mutagenic |
| | distilled water | | |

TABLE 2
Results of Ames mutagenicity test of L. speciosa, E. hirta,
and z. officinale on Salmonella typhimurium strain T100

| | | No. of Revernant | |
|---|---|---|---|
| Sample | Concentration | Colonies per plate | Mutagenicity |
| Positive control | 1.5 μg/ml | 161.4 ± 9.26 | Mutagenic |
| (Methyl methane | | | |
| sulfonate) | | | |
| Spontaneous control | — | 116.2 ± 7.29 | Non-mutagenic |
| Negative control | As is | 119.2 ± 10.23 | Non-mutagenic |
| (purified distilled | | | |
| water) | | | |
| E. hirta | 250 mg/ml in | 116.0 ± 9.67 | Non-mutagenic |
| | distilled water | | |
| L. speciosa | 250 mg/ml in | 118.0 ± 7.25 | Non-mutagenic |
| | distilled water | | |
| Z. officinale | 250 mg/ml in | 117.0 ± 10.56 | Non-mutagenic |
| | distilled water | | |

TABLE 3
Results of Micronucleus test of L. speciose,
E. hirta, and Z. officinale

| | | | No. of | |
|---|---|---|---|---|
| | | | MNPCEs per | |
| | | | 1000 PCEs + | |
| Sample | Concentration | | SEM | Mutagenicity |
| Positive control | 55 mg/kg | mouse | 2.07 ± 0.38 | Mutagenic |
| (Tetracycline) | | | | |
| Spontaneous | — | | 0.60 ± 0.15 | Non-mutagenic |
| control | | | | |
| Solvent control | 0.2 ml/20 g | mouse | 0.73 ± 0.13 | Non-mutagenic |
| (distilled water) | | | | |

| | | | |
|---|---|---|---|
| E. hirta | 250 mg/ml in | 0.73 ± 0.12 | Non-mutagenic |
| | distilled water | | |
| L. speciosa | 250 mg/ml in | 0.67 ± 0.19 | Non-mutagenic |
| | distilled water | | |
| Z. officinale | 250 mg/ml in | 0.67 ± 0.19 | Non-mutagenic |
| | distilled water | | |

The acute toxicity of the herbal formulations were determined in female Sprague-Dawley rats based on the procedure described in the Organization for Economic Cooperation and Development (OECD) Guideline 425: Up and Down Method. It included the calculation of the LD50 (the dose resulting to 50% mortality), cage side observations (body weight and temperature, fur characteristics, ophthalmologic findings, central nervous system—autonomic and cardiovascular manifestations), as well as gross and histopathologic examinations to monitor or verify any sign of toxicity at the tested dose levels. The results showed that the dried-pulverized L. speciosa, Z. officinale, E. hirta plant material and their 1:1:1 combination were found to be non-lethal at 2000 mg/Kg BW. This renders the plants tested under the Globally Harmonized System level 5 for safety classification as per definition of OECD. No significant signs of toxicity were seen during the short term and long term observation period. Blood analysis also revealed no significant differences in clinical parameters across groups with the exception of total protein, phosphorous, potassium, and chloride levels. Hematological data suggest that only in WBC, MCV and MCHC that the effects of treatments were significant. Those treated with L. speciosa have significantly less WBC and MCV as compared with the other four groups. On the contrary, their MCHC is significantly less than the other four groups. Microscopic analysis of tissues showed no significant diagnostic abnormalities that can be attributed to the plant materials tested. The results of such tests are attached as supplementary documents.

Meanwhile, the subchronic toxicity of the products will be determined in male and female Sprague-Dawley rats using the methods described in US FDA Red Book Subchronic Toxicity Testing IV C4. It included cage side observations, hematology, clinical chemistry, urinalysis, as well as gross and histopathologic examinations to monitor or verify any sign of toxicity at the tested dose levels. Cage side observations and necropsy of the vital organs of the treated rats with L. speciose, E. hirta. Z. officinale, and their 1:1:1 combination did not indicate any significant sample related signs of toxicity. Observed changes in weight and food consumption are attributed to the normal growth phase of the animals. Some of the changes in the hematological, clinical chemistry, and urinalysis parameters of the low, middle, and high doses of the plant samples are statistically significant but not drastic as compared to their respective baseline and control values. Data suggests that the oral administration of the mentioned samples at doses of 250 mg/kg. 500 mg/Kg. and 1000 mg/Kg BW did not cause any significant toxicity in both female and male Sprague Dawley rat from Day O up to the 90^{th} day of dosing. The results of the physiological and biochemical diagnostics test to assess the potential health risk of the daily intake of the mentioned samples on rats indicate the possible safety of the daily consumption of these plants in humans. The results of such tests are attached as supplementary documents.

An interesting positive result of the sub-chronic toxicity study is the remarkable elevation of the blood platelet count in both female and male rats as compared to the control treatment as presented in Tables 4-11 below. Such pre-clinical evidence serves as proof of concept in the efficacy of the mentioned plant samples in elevating blood platelet levels which in turn is crucial in the treatment of dengue-induced thrombocytopenia.

TABLE 4
Changes in the platelet count (×10\hat{ }9/L) for female rat test subjects of L. speciosa
Platelet Count (×10\hat{ }9/L)

| Group | Baseline | Day 30 | Day 60 | Day 90 |
|---|---|---|---|---|
| 250 mg/Kg L. speciosa | 273.00 ± 50.46 | 675.00 ± 70.87* | 835.00 ± 63.35* | 911.40 ± 43.50^{†}* |
| 500 mg/Kg L. speciosa | 456.80 ± 106.7 | 680.40 ± 65.85 | 859.40 ± 54.34* | 902.20 ± 96.10^{†}* |
| 1000 mg/Kg L. speciosa | 355.00 ± 47.09 | 1040.8 ± 56.68^{†}* | 1008.6 ± 45.96^{†}* | 969.40 ± 118.75^{†}* |
| Control | 258.80 ± 38.58 | 523.40 ± 89.98 | 706.60 ± 49.97* | 435.60 ± 125.72 |

Value expressed as mean ± SEM;
*p < 0.05 compared with the baseline;
^{†}p < 0.05 compared with the control.

TABLE 5
Changes in the platelet count (×10\hat{ }9/L) for male rat test subjects of L. speciosa
Platelet Count (×10\hat{ }9/L)

| Group | Baseline | Day 30 | Day 60 | Day 90 |
|---|---|---|---|---|
| 250 mg/Kg L. speciosa | 469.20 ± 56.01^{†} | 489.40 ± 28.37^{†} | 852.40 ± 40.29* | 816.80 ± 44.82* |
| 500 mg/Kg L. speciosa | 537.8 ± 66.53^{†} | 758.00 ± 59.81 | 846.20 ± 35.81* | 846.40 ± 47.82* |
| 1000 mg/Kg L. speciosa | 490.00 ± 68.13^{†} | 979.60 ± 23.88^{†}* | 800.00 ± 80.71* | 995.80 ± 62.21^{†}* |
| Control | 187.00 ± 49.46 | 707.40 ± 28.42* | 718.40 ± 20.24* | 602.60 ± 103.3* |

Value expressed as mean ± SEM;
*p < 0.05 compared with the baseline;
^{†}p < 0.05 compared with the control.

TABLE 6
Changes in the platelet count (×10\hat{ }9/L) for female rat test subjects of E. hirta
Platelet Count (×10\hat{ }9/L)

| Group | Baseline | Day 30 | Day 60 | Day 90 |
|---|---|---|---|---|
| 250 mg/Kg E. hirta | 329.80 ± 47.56 | 582.00 ± 54.31* | 887.80 ± 21.92* | 914.6 ± 19.94^{†}* |
| 500 mg/Kg E. hirta | 419.20 ± 116.7 | 804.80 ± 45.11 | 975.60 ± 103.75* | 1113.0 ± 68.12^{†}* |
| 1000 mg/Kg E. hirta | 584.20 ± 54.76 | 1196.6 ± 133.8^{†}* | 1242.8 ± 72.69^{†}* | 1226.8 ± 24.72^{†}* |
| Control | 258.80 ± 38.58 | 523.40 ± 89.98 | 706.60 ± 49.97* | 435.60 ± 125.72 |

Value expressed as mean ± SEM;
*p < 0.05 compared with the baseline;
^{†}p < 0.05 compared with the control.

TABLE 7
Changes in the platelet count (×10\hat{ }9/L) for male rat test subjects of E. hirta
Platelet Count (×10\hat{ }9/L)

| Group | Baseline | Day 30 | Day 60 | Day 90 |
|---|---|---|---|---|
| 250 mg/Kg E. hirta | 538.20 ± 87.74^{†} | 767.80 ± 22.04* | 865.00 ± 35.34* | 913.20 ± 21.24^{†}* |
| 500 mg/Kg E. hirta | 547.80 ± 73.78^{†} | 879.20 ± 13.46^{†}* | 787.60 ± 72.25 | 1111.40 ± 53.49^{†}* |
| 1000 mg/Kg E. hirta | 531.40 ± 98.70 | 1075.40 ± 53.89^{†}* | 1044.80 ± 35.83^{†}* | 1272.0 ± 20.50^{†}* |
| Control | 187.00 ± 49.46 | 707.40 ± 28.42* | 718.40 ± 20.24* | 602.60 ± 103.3* |

Value expressed as mean ± SEM;
*p < 0.05 compared with the baseline;
^{†}p < 0.05 compared with the control.

TABLE 8
Changes in the platelet count (×10\hat{ }9/L) for female rat test subjects of Z. officinale
Platelet Count (×10\hat{ }9/L)

| Group | Baseline | Day 30 | Day 60 | Day 90 |
|---|---|---|---|---|
| 250 mg/Kg Z. officinale | 424.60 ± 20.05 | 814.00 ± 84.09 | 960.60 ± 58.23* | 679.00 ± 170.0 |
| 500 mg/Kg Z. officinale | 609.20 ± 76.70 | 812.00 ± 72.02 | 992.00 ± 59.27 | 512.60 ± 143.0 |
| 1000 mg/Kg Z. officinale | 465.40 ± 47.44 | 1399.60 ± 172.5*^{†} | 1434.40 ± 173.4*^{†} | 753.80 ± 174.6 |
| Control | 438.00 ± 44.86 | 725.00 ± 96.34 | 662.80 ± 164.3 | 548.00 ± 146.0 |

Value expressed as mean ± SEM;
*p < 0.05 compared with the baseline;
^{†}p < 0.05 compared with the control.

TABLE 9
Changes in the platelet count (×10\hat{ }9/L) for male rat test subjects of Z. officinale
Platelet Count (×10\hat{ }9/L)

| Group | Baseline | Day 30 | Day 60 | Day 90 |
|---|---|---|---|---|
| 250 mg/Kg Z. officinale | 592.60 ± 58.49 | 607.20 ± 34.91^{†} | 968.80 ± 44.87* | 1023.40 ± 85.02* |
| 500 mg/Kg Z. officinale | 663.60 ± 67.44^{†} | 877.00 ± 57.37 | 960.40 ± 37.41 | 856.00 ± 177.5 |
| 1000 mg/Kg Z. officinale | 520.00 ± 51.11 | 1105.40 ± 11.40*^{†} | 918.20 ± 87.79 | 863.00 ± 171.9 |
| Control | 379.80 ± 35.62 | 829.00 ± 33.43* | 879.00 ± 34.10* | 950.40 ± 143.6* |

TABLE 10
Changes in the platelet count (×10\hat{ }9/L) for female rat test subjects
of 1:1:1 Combination of the L speciosa, E. hirta, and Z. officinale
Platelet Count (×10\hat{ }9/L)

| Group | Baseline | Day 30 | Day 60 | Day 90 |
|---|---|---|---|---|
| 250 mg/KgCombination | 447.20 ± 56.83 | 725.00 ± 96.34 | 662.80 ± 164.3* | 548.00 ± 146.03 |
| 500 mg/Kg Combination | 526.40 ± 114.2 | 940.60 ± 54.90 | 1086.00 ± 97.63 | 716.60 ± 222.7 |
| 1000 mg/Kg Combination | 498.00 ± 50.04 | 1309.80 ± 53.29*^{†} | 1418.40 ± 127.3*^{†} | 709.80 ± 181.2 |
| Control | 438.00 ± 44.86 | 725.00 ± 96.34 | 662.80 ± 164.3 | 548.00 ± 146.0 |

Value expressed as mean ± SEM;
*p < 0.05 compared with the baseline;
^{†}p < 0.05 compared with the control.

TABLE 11
Changes in the platelet count (×10\hat{ }9/L) for male rat test subjects
of 1:1:1 Combination of the L. speciosa, E. hirta, and Z. officinale
Platelet Count (×10\hat{ }9/L)

| Group | Baseline | Day 30 | Day 60 | Day 90 |
|---|---|---|---|---|
| 250 mg/KgCombination | 611.80 ± 95.62 | 853.00 ± 29.50 | 964.00 ± 35.10 | 822.80 ± 207.6 |
| 500 mg/Kg Combination | 611.00 ± 68.26 | 970.20 ± 19.96 | 888.60 ± 81.85 | 931.60 ± 176.1 |
| 1000 mg/Kg Combination | 620.00 ± 97.26 | 1191.80 ± 66.21*^{†} | 1144.80 ± 33.35*^{†} | 914.80 ± 78.52 |
| Control | 379.80 ± 35.62 | 829.00 ± 33.43* | 879.00 ± 34.10* | 950.40 ± 143.6* |

Value expressed as mean ± SEM;
*p < 0.05 compared with the baseline;
^{†}p < 0.05 compared with the control.

Another important proof of concept is the ability of the mentioned plant samples in inhibiting replication of dengue virus (DV). This was tested by Plaque Reduction Neutralization Test (PRNT) which is considered as the “gold standard” in characterizing and quantifying circulating levels of anti-DV neutralizing antibody (NAb)23. The results in Table 12 showed that all plant samples and their combinations have remarkable inhibitory effect on dengue virus infection as reflected in their drastic reduction of plaque formation in dengue-infected Vero cells. E. hirta, L. speciosa, and their 1:1 combination showed the most potent inhibitory action both on DV1 and DV3.

TABLE 12
Summary of DV1 and DV3 Plaque Reduction of L. speciosa, E. hirta,
Z. officinale, and their combinations as tested on Vero cells.

| | Average Number of Plaques | |
|---|---|---|
| Test Samples | DV1 infected cells | DV3 infected cells |
| Virus alone | >100 | >100 |
| Z. officinale | 38.0 | 35.0 |
| E. hirta | 0.0 | 0.0 |
| L. speciosa | 0.0 | 2.3 |
| E hirta + L. speciosa | 1.3 | 0.0 |
| E. hirta + L. speciosa + | 22.7 | 4.0 |
| Z. officinale | | |

Clinical Studies

Safety and Dose Escalation Trial of the herbal formulation will be conducted in healthy human subjects under fasting conditions using the methods as described. In addition, patients with dengue consulting with partner hospitals will be given two 500 mg capsules of the formulated herbal drug every 3 hours for 3 days and the alleviation of platelet depletion and hemovascular instability, duration of illness and mortality as endpoints will be determined and compared to the placebo.

1. A herbal medicine composition, comprising:
a dried and finely powdered leaves of Lagerstroemia speciosa;
b dried and finely powdered aerial parts of Euphorbia hirta; and
c dried and finely powdered rhizome of Zingiber officinale;
wherein the herbal medicine composition comprises a fixed-dose combination of two or three of the components a, b and c.

2. The herbal medicine composition according to claim 1, wherein the components a, b and c are present in spray-dried aqueous, ethanolic, methanolic or hydroalcoholic solvent, 70-95% ethanol and/or methanol by volume.

3. A method for producing the herbal medicine composition of claim 1 the method comprising:
a preparing dried raw plant materials by air-drying and/or with a commercial dehydrator to reduce a moisture content of raw plant materials to below 10%;
powdering the dried raw pant materials with a commercial blender or mill, thereby obtaining powdered dried materials;
sieving the powdered dried materials through No. 60 mesh or finer sieve, thereby obtaining processed materials; and
encapsulating the processed materials with a suitable capsule.

4. The method of claim 2, further comprising:
extracting an extract of the processed materials by an exhaustive cold maceration with a distilled water, ethanol, methanol or a hydroalcoholic solvent (70%-95% ethanol and/or methanol by volume);
spray-drying of the extract thereof, thereby obtaining sprayed-dried extract; and
encapsulating the sprayed-dried extract with a suitable capsule.

5. The herbal medicine composition of claim 1, where all three plant materials a, b and c are present in a fixed dose combination and
a ratio of the three plant materials optionally vary but A total combined dosage is 500 mg and 400 mg.
